(19) United States
(12) Patent Application Publication
(10) Pub. No.: US 2011/0060210 A1
(43) Pub. Date: Mar. 10, 2011

(54) METHOD FOR ASSESSING THE PROBABILITY OF DISEASE DEVELOPMENT IN TISSUE

(76) Inventor: Richard L. Ehman, Rochester, MN (US)

(21) Appl. No.: 12/990,859

(22) PCT Filed: May 1, 2009
(86) PCT No.: PCT/US09/42542

§ 371 Date: Nov. 3, 2010

Related U.S. Application Data
(60) Provisional application No. 61/050,304, filed on May 5, 2008.

(51) Int. Cl. A61B 5/055; A61B 5/00

(52) U.S. Cl. 600/410; 600/587

(57) ABSTRACT

A method for detecting conditions in tissues that can lead to disease includes an in vivo measurement of the mechanical characteristics of the tissues using Magnetic Resonance Elastography (MRE). The deviation of mechanical characteristics such as tissue stiffness from a predetermined norm is determined and indicated on an image of the tissues.

### BACKGROUND OF THE INVENTION

The physician has many diagnostic tools at his or her disposal which enable detection and localization of diseased tissues. These include x-ray systems that measure and produce images indicative of the x-ray attenuation of the tissues and ultrasound systems that detect and produce images indicative of tissue echogenicity and the boundaries between structures of differing acoustic properties. Nuclear medicine produces images indicative of those tissues which absorb tracers injected into the patient, as do PET scanners and SPECT scanners. And finally, magnetic resonance imaging (“MRI”) systems produce images indicative of the magnetic properties of tissues. It is fortuitous that many diseased tissues are detected by the physical properties measured by these imaging modalities, but it should not be surprising that many diseases go undetected.

Historically, one of the physician's most valuable diagnostic tools is palpation. By palpating the patient a physician can feel differences in the compliance or “stiffness”, of tissues and detect the presence of tumors and other tissue abnormalities. Unfortunately, this valuable diagnostic tool is limited to those tissues and organs which the physician can feel, and many diseased internal organs go undiagnosed unless the disease happens to be detectable by one of the above imaging modalities. Tumors (e.g. of the liver) that are undetected by existing imaging modalities and cannot be reached for palpation through the patient's skin and musculature, are often detected by surgeons by direct palpation of the exposed organs at the time of surgery. Palpation is the most common means of detecting tumors of the prostate gland and the breast, but unfortunately, deeper portions of these structures are not accessible for such evaluation.

It has been found that MR imaging can be enhanced when an oscillating stress is applied to the object being imaged in a method called MR elastography (MRE). The method requires that the oscillating stress produce shear waves that propagate through the organ, or tissues to be imaged. These shear waves alter the phase of the MR signals, and from this the mechanical properties of the subject can be determined. In many applications, the production of shear waves in the tissues is merely a matter of physically vibrating the surface of the subject with an electromechanical device.

As disclosed in U.S. Pat. No. 5,825,186 MRI methods are known for producing images in which the image contrast is modulated by tissue stiffness. Other mechanical properties of tissues can also be measured in vivo as described in U.S. Pat. No. 5,592,085. This method is known as magnetic resonance elastography (MRE) and it has been used successfully to image and detect liver fibrosis.

Other methods for in vivo measurement of the mechanical properties of tissues are known. One such method is referred to as vibro-acoustography. As described in M. Fatemi and J. F. Greenleaf in their publication “Vibro-Acoustography: An Imaging Modality on Ultrasound-Stimulated Acoustic Emission”, Proc. Natl. Acad. Sci. USA, Vol. 96, pp. 6603-08, June 1999 Engineering, this method applies an oscillatory force to tissues and measures the acoustic emission field with an ultrasound scanner. From the measured acoustic emissions the mechanical characteristics of the tissues can be determined.

Many disease processes cause marked changes in the mechanical properties of tissue. For instance, hepatic fibrosis causes increased stiffness of liver tissue, and many benign and malignant tumors are harder, or stiffer than surrounding normal tissues. This has provided motivation for development of methods for quantitatively mapping the mechanical properties of tissues in the body for diagnostic purposes. These developments have focused on diagnosing disease by detecting the changes in tissue mechanical properties that are caused by the disease process. In all cases, such in vivo methods detect the presence of disease after the disease is fully manifested.

In the field of cell biology, there has been a growing awareness of the importance of tissue matrix mechanics on cellular function in natural and engineered tissues. Cells are known to sense their mechanical environment through myosin-based contractility of the cytoskeleton in conjunction with adhesion molecules such as integrins and cadherins. Cells react to the dynamic and static properties of their matrix environment through mechanotransduction and cytoskeletal remodeling, Discher D E, Janmey P, Wang, Y L. Tissue cells feel and respond to the stiffness of their substrate. Science 2005;310(5751):1139-1143.

There is increasing interest in assessing the mechanical properties of the matrix environment, given its profound influence on the behavior of cells in diverse areas such as morphogen-mediated cell programming and differentiation in developing embryos, Pelham R J, Wang Y L. Cell locomotion and focal adhesions are regulated by substrate flexibility. Proceedings of the National Academy of Sciences of the United State of America 1997;94(25):13661-13665; Georges P C, Janmey P A. Cell type-specific response to growth on soft materials. Journal of Applied Physiology 2005; 98(4):1547-1553; Saez A, Ghibaudo M, Buguin A, Silberzan P, Ladoux B. Rigidity-driven growth and migration of epithelial cells on microstructured anisotropic substrates. Proceedings of the National Academy of Sciences of the United States of America 2007;104(20):8281-8286, activation of hepatic stellate cells to initiate liver fibrosis, Wells R G. The role of matrix stiffness in hepatic stellate cell activation and liver fibrosis. Journal of Clinical Gastroenterology 2005;39:S158-S161; Sakata R, Ueno T, Makamura T, Ueno H, Sata M. Mechanical stretch induces TGF-beta synthesis in hepatic stellate cells. European Journal of Clinical Investigation 2004;34(2):129-136; Georges P C, Jui J J, Gombos Z, McCormick M E, Wang A Y, Uemura M, Mick R, Janmey P A, Furth E E, Wells R G. Increased stiffness of the rat liver precedes matrix deposition: implications for fibrosis. American Journal of Physiology-Gastrointestinal and Liver Physiology 2007; 293(6):LG1147-G1154, regulation of ovarian follicular function, West E R, Xu M, Woodruff T K, Shea L D. Physical properties of alginate hydrogels and their effects on in vitro follicle development. Biomaterials 2007;28(30):4439-4448, and dell behavior in engineered tissue constructs, Fedorovich N E, Alblas J, de Wijn J R, Hennink W E, Verbout A J, Dhert W J A. Hydrogels as extracellular matrices for skeletal tissue engineering: state-of-the-art and novel application in organ printing. Tissue Engineering 2007;13(8):1905-1925. Recent research has also shown that increased matrix stiffness perturbs epithelial morphogenesis through integrins to increase cellular contractility and rigidity and there is strong evidence that this process drives the onset of malignant transformation in some tissues, Paszek M J, Zahir N, Johnson K R, Lakins J N, Rozenberg G I, Gefen A, Reinhart-King C A, Margulies S S, Dembo M, Boettiger D, Hammer D A, Weaver V M. Tensional homeostasis and the malignant phenotype. Cancer Cell 2005;8(3):241-254.

### SUMMARY OF THE INVENTION

The present invention is a method for detecting conditions in tissues that can lead to the development of certain disease states. It is known that abnormal tissue mechanical properties can be a significant cause of certain disease processes, and thus the detection of such abnormal tissue properties can provide a way to predict the development of certain disease states. By detecting such conditions, the present invention enables one to take preemptive actions that terminate or mitigate the conditions, thereby preventing the disease before it develops.

The present invention includes establishing a mechanical characteristic in tissues that can lead to a disease condition in such tissues, performing an in vivo measurement of the tissue mechanical characteristic in a subject, determining whether the measured mechanical characteristic is present in the subject tissues, and indicating the disease producing condition in the subject tissues. Possible mechanical characteristics include such properties as stiffness, elasticity, viscosity, shear attenuation and stretch. These characteristics can be measured in vivo using quantitative or semi-quantitative techniques such as dynamic MR elastography, acoustic radiation force elastography, acoustic vibrometry elastography and transient ultrasound elastography. Static and quasi-static measurement techniques may also be used such as ultrasound strain imaging, acoustic radiation force strain imaging, acoustic vibrometry, as well as indentation devices such as durometers.

In one preferred embodiment of the invention abnormal mechanical properties can be detected using an MRE imaging method that measures the mechanical properties of tissues in vivo. A disease producing condition may be indicated by the absolute value of a measured mechanical property such as stiffness, or when a series of MRE images are acquired over time, a disease producing condition may be indicated by a change in a measured mechanical property such as stiffness. Such a condition may be detected long before the disease develops and while other diagnostic procedures, such as biopsy and microscopic evaluation of the tissue would reveal no abnormality.

An object of the invention is to identify subjects who are at an increased risk for tumor development. Certain conditions in the extracellular matrix of tissues that are identifiable with MRE, such as increased mechanical tension across cells or increased stiffness will increase the probability of malignant transformation. Therefore patients who are found to have such changes in the mechanical properties of otherwise normal tissue may be at much greater risk for eventual development of cancer in those tissues. If such changes are detected, appropriate measures may be prescribed to reverse the changes and thereby prevent the development of cancer or, if this is not feasible, to implement more frequent diagnostic surveillance to detect and treat tumors at an early stage.

Another object of the invention is to identify subjects who are at increased risk for development of organ fibrosis or other diseases that are potentiated by changes in the mechanical properties of the environment surrounding tissue cells. The detection of organ fibrosis is typically done ex-vivo using an extracted sample of the tissue, although in vivo detection methods using MRE are becoming more common as described by Yin M, Talwalkar J A, Glaser K J, Manduca A, Grimm R C, Rossman P J, Fidler J L, Ehman R L. “Assessment of hepatic fibrosis with magnetic resonance elastography” Clinical Gastroenterology & Hepatology. 2007; 5(10):1207-1213. The present invention goes a step further and measures conditions in the organ that can lead to fibrosis. This enables physicians to prescribe actions that can prevent fibrosis and organ damage before it even develops.

The foregoing and other objects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a block diagram of an NMR system which is employed to practice a preferred embodiment of the present invention;

FIG. 2 is an electrical block diagram of the transceiver which forms part of the NMR system of FIG. 1;

FIG. 3 is a graphic representation of a pulse sequence performed by the NMR system of FIG. 1 to practice the preferred embodiment of the invention; and

FIG. 4 is a flow chart which indicates the steps employed in accordance with the preferred embodiment of the invention.

### DETAILED DESCRIPTION OF THE PREFERRED EMBODIMENT

As indicated above, the present invention may be implemented using any of a number of different techniques for the in vivo measurement of the mechanical characteristics of the tissues of interest. In many cases the choice will be determined by the particular tissues being examined or the particular equipment that is available. Similarly, the particular mechanical characteristic that is measured will be determined not only by the particular tissue being examined, but by the particular disease process of interest. In the preferred embodiment an MRE technique is used to detect the stiffness in organ tissues as a means for predicting the possible onset of fibrosis.

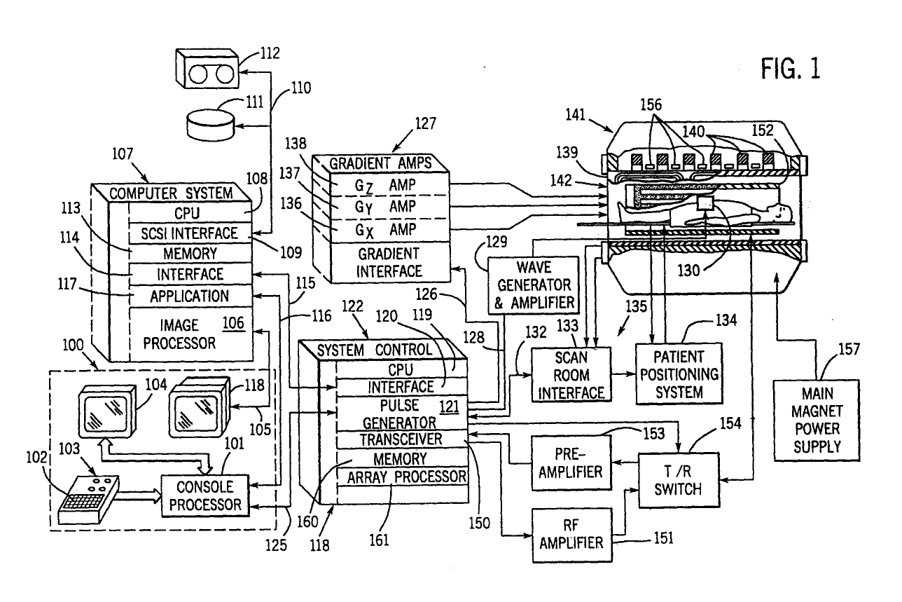

Referring first to FIG. 1, there is shown the major components of a preferred NMR system which incorporates the present invention and which is sold by the General Electric Company under the trademark “SIGNA”. The operation of the system is controlled from an operator console 100 which includes a console processor 101 that scans a keyboard 102 and receives inputs from a human operator through a control panel 103 and a plasma display/touch screen 104. The console processor 101 communicates through a communications link 116 with an applications interface module 117 in a separate computer system 107. Through the keyboard 102 and controls 103, an operator controls the production and display of images by an image processor 106 in the computer system 107, which connects directly to a video display 118 on the console 100 through a video cable 105.

The computer system 107 includes a number of modules which communicate with each other through a backplane. In addition to the application interface 117 and the image processor 106, these include a CPU module 108 that controls the backplane, and an SCSI interface module 109 that connects the computer system 107 through a bus 110 to a set of peripheral devices, including disk storage 111 and tape drive 112. The computer system 107 also includes a memory module 113, known in the art as a frame buffer for storing image data arrays, and a serial interface module 114 that links the computer system 107 through a high speed serial link 115 to a system interface module 120 located in a separate system control cabinet 122.

The system control 122 includes a series of modules which are connected together by a common backplane 118. The backplane 118 is comprised of a number of bus structures, including a bus structure which is controlled by a CPU module 119. The serial interface module 120 connects this backplane 118 to the high speed serial link 115, and pulse generator module 121 connects the backplane 118 to the operator console 100 through a serial link 125. It is through this link 125 that the system control 122 receives commands from the operator which indicate the scan sequence that is to be performed.

The pulse generator module 121 operates the system components to carry out the desired scan sequence. It produces data which indicates the timing, strength and shape of the RF pulses which are to be produced, and the timing of and length of the data acquisition window. The pulse generator module 121 also connects through serial link 126 to a set of gradient amplifiers 127, and it conveys data thereto which indicates the timing and shape of the gradient pulses that are to be produced during the scan.

In the preferred embodiment of the invention the pulse generator module 121 also produces sync pulses through a serial link 128 to a wave generator and amplifier 129. The wave generator produces a sinusoidal voltage which is synchronized to the frequency and phase of the received sync pulses and this waveform is output though a 50 watt, dc coupled audio amplifier. A frequency in the range of 20 Hz to 1000 Hz is produced depending on the particular object being imaged, and it is applied to a transducer 130. The transducer 130 will be described in more detail below, and its structure depends on the particular anatomy being measured and imaged. In general, however, the transducer 130 produces a force, or pressure, which oscillates in phase with the sync pulses produced by the pulse generator module 121 and creates an oscillating stress in the gyromagnetic media (i.e. tissues) to which it is applied.

And finally, the pulse generator module 121 connects through a serial link 132 to scan room interface circuit 133 which receives signals at inputs 135 from various sensors associated with the position and condition of the patient and the magnet system. It is also through the scan room interface circuit 133 that a patient positioning system 134 receives commands which move the patient cradle and transport the patient to the desired position for the scan.

The gradient waveforms produced by the pulse generator module 121 are applied to a gradient amplifier system 127 comprised of Gx, Gy and Gz amplifiers 136, 137 and 138, respectively. Each amplifier 136, 137 and 138 is utilized to excite a corresponding gradient coil in an assembly generally designated 139. The gradient coil assembly 139 forms part of a magnet assembly 141 which includes a polarizing magnet 140 that produces either a 0.5 or a 1.5 Tesla polarizing field that extends horizontally through a bore 142. The gradient coils 139 encircle the bore 142, and when energized, they generate magnetic fields in the same direction as the main polarizing magnetic field, but with gradients Gx, Gy and Gz directed in the orthogonal x-, y- and z-axis directions of a Cartesian coordinate system. That is, if the magnetic field generated by the main magnet 140 is directed in the z direction and is termed B0, and the total magnetic field in the z direction is referred to as Bz, then Gx=ÿBz/ÿx, Gy=ÿBz/ÿy and Gz=ÿBz/ÿz, and the magnetic field at any point (x,y,z) in the bore of the magnet assembly 141 is given by B(x,y,z)=B0+Gxx+Gyy+Gzz. The gradient magnetic fields are utilized to encode spatial information into the NMR signals emanating from the patient being scanned, and as will be described in detail below, they are employed to measure the microscopic movement of spins caused by the pressure produced by the transducer 130.

Located within the bore 142 is a circular cylindrical whole-body RF coil 152. This coil 152 produces a circularly polarized RF field in response to RF pulses provided by a transceiver module 150 in the system control cabinet 122. These pulses are amplified by an RF amplifier 151 and coupled to the RF coil 152 by a transmit/receive switch 154 which forms an integral part of the RF coil assembly. Waveforms and control signals are provided by the pulse generator module 121 and utilized by the transceiver module 150 for RF carrier modulation and mode control. The resulting NMR signals radiated by the excited nuclei in the patient may be sensed by the same RF coil 152 and coupled through the transmit/receive switch 154 to a preamplifier 153. The amplified NMR signals are demodulated, filtered, and digitized in the receiver section of the transceiver 150. The transmit/receive switch 154 is controlled by a signal from the pulse generator module 121 to electrically connect the RF amplifier 151 to the coil 152 during the transmit mode and to connect the preamplifier 153 during the receive mode. The transmit/receive switch 154 also enables a separate RF coil (for example, a head coil or surface coil) to be used in either the transmit or receive mode.

In addition to supporting the polarizing magnet 140 and the gradient coils 139 and RF coil 152, the main magnet assembly 141 also supports a set of shim coils 156 associated with the main magnet 140 and used to correct inhomogeneities in the polarizing magnet field. The main power supply 157 is utilized to bring the polarizing field produced by the superconductive main magnet 140 to the proper operating strength and is then removed.

The NMR signals picked up by the RF coil 152 are digitized by the transceiver module 150 and transferred to a memory module 160 which is also part of the system control 122. When the scan is completed and an entire array of data has been acquired in the memory module 160, an array processor 161 operates to Fourier transform the data into an array of image data. This image data is conveyed through the serial link 115 to the computer system 107 where it is stored in the disk memory 111. In response to commands received from the operator console 100, this image data may be archived on the tape drive 112, or it may be further processed by the image processor 106 as will be described in more detail below and conveyed to the operator console 100 and presented on the video display 118.

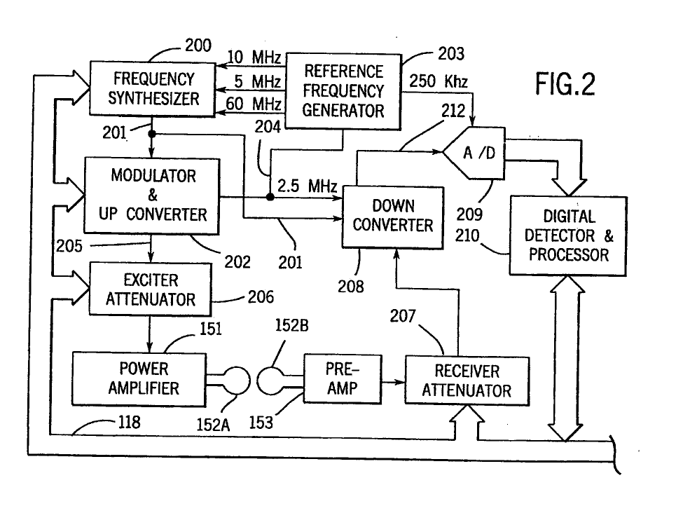

Referring particularly to FIGS. 1 and 2, the transceiver 150 includes components which produce the RF excitation field B1 through power amplifier 151 at a coil 152A and components which receive the resulting NMR signal induced in a coil 152B. As indicated above, the coils 152A and B may be separate as shown in FIG. 2, or they may be a single wholebody coil as shown in FIG. 1. The base, or carrier, frequency of the RF excitation field is produced under control of a frequency synthesizer 200 which receives a set of digital signals (CF) through the backplane 118 from the CPU module 119 and pulse generator module 121. These digital signals indicate the frequency and phase of the RF carrier signal which is produced at an output 201. The commanded RF carrier is applied to a modulator and up converter 202 where its amplitude is modulated in response to a signal R(t) also received through the backplane 118 from the pulse generator module 121. The signal R(t) defines the envelope, and therefore the bandwidth, of the RF excitation pulse to be produced. It is produced in the module 121 by sequentially reading out a series of stored digital values that represent the desired envelope. These stored digital values may, in turn, be changed from the operator console 100 to enable any desired RF pulse envelope to be produced. The modulator and up converter 202 produces an RF pulse at the desired Larmor frequency at an output 205.

The magnitude of the RF excitation pulse output through line 205 is attenuated by an exciter attenuator circuit 206 which receives a digital command, TA, from the backplane 118. The attenuated RF excitation pulses are applied to the power amplifier 151 that drives the RF coil 152A.

Referring still to FIGS. 1 and 2 the NMR signal produced by the subject is picked up by the receiver coil 1528 and applied through the preamplifier 153 to the input of a receiver attenuator 207. The receiver attenuator 207 further amplifies the NMR signal and this is attenuated by an amount determined by a digital attenuation signal (RA) received from the backplane 118. The receive attenuator 207 is also turned on and off by a signal from the pulse generator module 121 such that it is not overloaded during RF excitation.

The received NMR signal is at or around the Larmor frequency, which in the preferred embodiment is around 63.86 MHz for 1.5 Tesla and 21.28 MHz for 0.5 Tesla. This high frequency signal is down converted in a two step process by a down converter 208 which first mixes the NMR signal with the carrier signal on line 201 and then mixes the resulting difference signal with the 2.5 MHz reference signal on line 204. The resulting down converted NMR signal on line 212 has a maximum bandwidth of 125 kHz and it is centered at a frequency of 187.5 kHz. The down converted NMR signal is applied to the input of an analog-to-digital (A/D) converter 209 which samples and digitizes the analog signal at a rate of 250 kHz. The output of the ND converter 209 is applied to a digital detector and signal processor 210 which produce 16-bit in-phase (I) values and 16-bit quadrature (Q) values corresponding to the received digital signal. The resulting stream of digitized I and Q values of the received NMR signal is output through backplane 118 to the memory module 160 where they are employed to reconstruct an image.

To preserve the phase information contained in the received NMR signal, both the modulator and up converter 202 in the exciter section and the down converter 208 in the receiver section are operated with common signals. More particularly, the carrier signal at the output 201 of the frequency synthesizer 200 and the 2.5 MHz reference signal at the output 204 of the reference frequency generator 203 are employed in both frequency conversion processes. Phase consistency is thus maintained and phase changes in the detected NMR signal accurately indicate phase changes produced by the excited spins. The 2.5 MHz reference signal as well as 5, 10 and 60 MHz reference signals are produced by the reference frequency generator 203 from a common 20 MHz master clock signal. The latter three reference signals are employed by the frequency synthesizer 200 to produce the carrier signal on output 201.

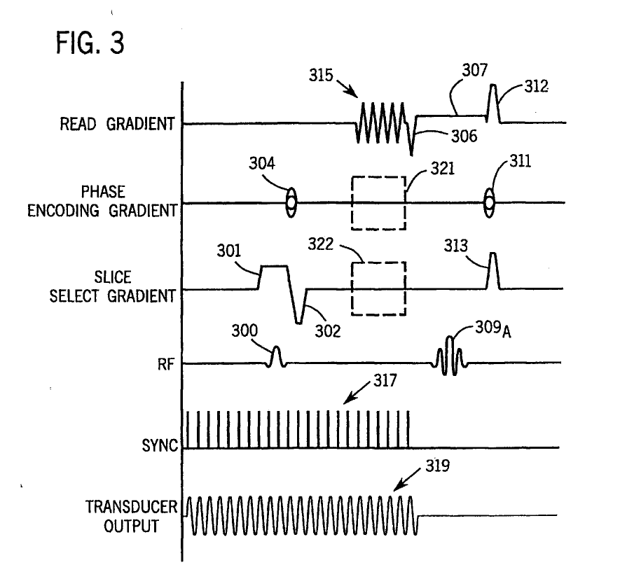

Referring particularly to FIG. 3, a preferred embodiment of a pulse sequence which may be used to acquire NMR data according to the present invention is shown. The pulse sequence is fundamentally a 2DFT pulse sequence using a gradient recalled echo. Transverse magnetization is produced by a selective 90° rf excitation pulse 300 which is produced in the presence of a slice select gradient (Gz) pulse 301 and followed by a rephasing gradient pulse 302. A phase encoding gradient (Gy) pulse 304 is then applied at an amplitude and polarity determined by the view number of the acquisition. A read gradient (Gx) is applied as a negative dephasing lobe 306, followed by a positive readout gradient pulse 307. An NMR echo signal 309 is acquired 40 msecs. after the rf excitation pulse 300 during the readout pulse 307 to frequency encode the 256 digitized samples. The pulse sequence is concluded with spoiler gradient pulses 312 and 313 along read and slice select axes, and a rephasing gradient pulse 311 is applied along the phase encoding axis (Gy). As is well known in the art, this rephasing pulse 311 has the same size and shape, but opposite polarity of the phase encoding pulse 304. The pulse sequence is repeated 128 times with the phase encoding pulse 304 stepped through its successive values to acquire a 128 by 256 array of complex NMR signal samples that comprise the data set (A).

To practice the present invention an alternating magnetic field gradient is applied after the transverse magnetization is produced and before the NMR signal is acquired. In the preferred embodiment illustrated in FIG. 3, the read gradient (Gx) is used for this function and is alternated in polarity to produce one or more bipolar, gradient waveforms 315. The alternating gradient 315 has a typical frequency of 60 Hz and a duration of 25 msecs. At the same time, the pulse generator module 121 produces sync pulses as shown at 317, which are also at a frequency of 60 Hz and have a specific phase relationship with the alternating gradient pulses 315. As explained above, these sync pulses 317 activate the transducer 130 to apply an oscillating stress 319 to the patient which has the same frequency and phase relationship. To insure that the resulting waves have time to propagate throughout the field of view, the sync pulses 317 may be turned on well before the pulse sequence begins, as shown in FIG. 3. Alternatively, the synch pulses and transducer motion may be applied continuously throughout the entire duration of data acquisition. In this embodiment, the repetition time of the MRI sequence is set to be an integral multiple of the period of the applied oscillating stress.

The phase of the NMR signal 309 is indicative of the movement of the spins. If the spins are stationary, the phase of the NMR signal is not altered by the alternating gradient pulses 315, whereas spins moving along the read gradient axis (x) will accumulate a phase proportional to the amplitude of the vibration. Spins which move in synchronism and in phase with the alternating magnetic field gradient 215 will accumulate maximum phase of one polarity, and those which move in synchronism, but 180° out of phase with the alternating magnetic field gradient 215 will accumulate maximum phase of the opposite polarity. The phase of the acquired NMR signal 309 is thus affected by the “synchronous” movement of spins along the x-axis.

The acquisition described in the preceeding section is typically repeated 4 times, each with a different phase relationship between the oscillating stress and the cyclic motion encoding gradient waveform. This is typically done by changing the timing relationship between the train of synch pulses and the initial RF excitation, 300. The 4 different acquisitons thereby provide views of the mechanical wave propagation pattern within the tissue at 4 equally-spaced times in the wave cycle.

The pulse sequence in FIG. 3 can be modified to measure synchronous spin movement along the other gradient axes (y and z). For example, the alternating magnetic field gradient pulses may be applied along the phase encoding axis (y) as indicated by dashed lines 321, or they may be applied along the slice select axis (z) as indicated by dashed lines 322. Indeed, they may be applied simultaneously to two or three of the gradient field directions to “read” synchronous spin movements along any desired direction.

The number of cycles of the alternating magnetic field gradient used in each pulse sequence depends on the strength of the applied gradient field, the frequency of the synchronous movement to be measured, and the TE time of the pulse sequence. The phase sensitivity of the pulse sequence to synchronous spin movement is proportional to the integrated product of alternating gradient field amplitude and the displacement over time. The sensitivity may be increased by increasing the amplitude of the gradient field pulses and by increasing the area under each pulse by making them as “square” as possible. The duration of each gradient pulse is limited by the desired synchronous frequency, and hence more cycles of the alternating gradient waveform are required at higher frequencies to produce the same sensitivity as a lower frequency alternating gradient of the same amplitude and wave shape.

In the preferred embodiment which measures the mechanical characteristics of the liver a transducer 130 such as that described in U.S. Pat. No. 7,034,534 is employed. It includes a passive diaphragm that is pressed against the subject's abdomen and which is vibrated by a remote electromagnetic driver that couples to the passive diaphragm via a flexible tube.

The oscillating stress may be applied by the transducer 130 in a number of ways. By starting the sync pulses 317 well before the alternating magnetic field gradient 315 as shown in FIG. 3, the synchronous spin motion propagates throughout the field of view of the reconstructed image. This will image the steady-state conditions in the medium when the oscillating stress is applied. If the sync pulses 317 are turned off just before the alternating gradient 315 is applied, spins adjacent to the transducer 130 are moving with less amplitude or not at all during the phase accumulation time period. This may be desired, for example, when regions deep beneath the surface are of primary interest and large strain effects in the image near the transducer 130 can be suppressed. If this is not a concern, then the oscillating stress may be applied continuously during the data acquisition.

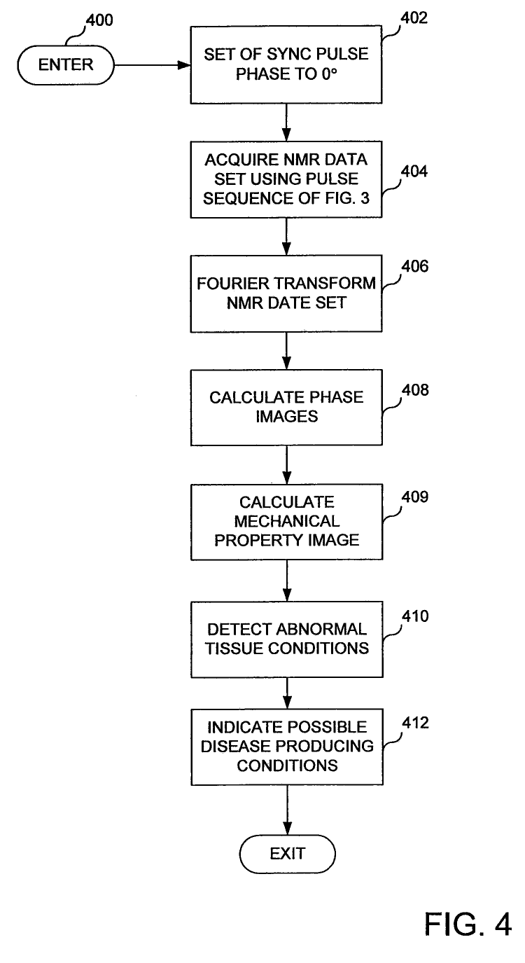

The preferred embodiment of the invention employs the MRI system to measure the stiffness of liver tissues in a subject who is predisposed to the development of progressive liver fibrosis. Prior studies have shown that subjects with liver stiffness values below 3 kPa are very unlikely to have detectable hepatic fibrosis. However, hepatic tissue stiffness values that are higher than the normal value of approximately 2 kPa, but below the upper normal limit of 3 kPa my indicate the presence of an altered mechanical environment to cells within the liver that could lead to eventual development of fibrosis and organ damage. Elevated tissue stiffness values are indicative of subtle changes in the macromolecular composition and structure of the extracellular space separating hepatocytes from hepatic sinusoids which can create a high potential for developing progressive fibrosis. Accordingly, stiffness in the upper part of the range between 2 kPa and 3 kPa is selected as the mechanical criterion which will indicate conditions in the liver that may lead to a disease.
A scan using the pulse sequence of FIG. 3 is carried out under the direction of a program executed by the NMR system of FIG. 1. Referring particularly to FIG. 4, a scan is performed according to the present invention to acquire NMR data from which the mechanical properties of the liver tissues can be measured. The program for this scan is entered at 400 and the pulse sequence of FIG. 3 is downloaded to the pulse generator module 121. The sync pulses 217 in this pulse sequence are timed to be in phase with the alternating motion encoding gradient 215 as indicated at process block 402. The pulse sequence is then performed the necessary number of times to acquire the complete NMR data set, as indicated at process block 404. This “k-space” NMR data set is then Fourier transformed at process block 406 along each of its two dimensions to produce an image data set. This is a complex Fourier transformation of the acquired quadrature signals I and Q to produce corresponding complex values I and Q at each pixel location in the image data set. As indicated at process block 408, the phase angle of the signal at each image pixel is calculated to generate an image depicting the pattern of propagating mechanical waves in the tissue.

As indicated at process block 409, the phase images depicting propagating waves are analyzed with a mathematical algorithm called an inversion, to generate images that can quantitatively display various mechanical properties of tissue as described by Manduca A, Oliphant T E, Dresner M A, et al. “Magnetic resonance elastography: in vivo non-invasive mapping of tissue elasticity”, Medical Imaging Analysis. 2001; 5(4): 237-254., and by Oliphant T E, Manduca A, Ehman R L, Greenleaf J F.“Complex-valued stiffness reconstruction for magnetic resonance elastography by algebraic inversion of the differential equation,” Magn Reson Med 2001; 45(2):299-310. This process typically involves phase unwrapping, fourier transformation of the pixel displacement values through the 4 times in the wave cycle to recover the wave information, and then application of a wavelength-estimating algorithm or a direct inversion of the wave equation algorithm to finally generate an image depicting a mechanical property such as tissue elastic modulus or stiffness.

As indicated in FIG. 4 at process block 410, the next step is to compare the measured mechanical properties values (in this case, stiffness) with the selected mechanical criteria indicative of a possible disease producing condition. In this embodiment, mean value of the stiffness of liver tissue is measured. In patients without liver fibrosis, if the average stiffness value is substantially higher than the normal mean value of 2.0 kPa, then this is often indicative of conditions in the extracellular matrix of the liver that promote the eventual development of liver fibrosis and scarring. For instance, a measurement of 2.8 kPa would indicate this condition. (The exact threshold to be used depends on the requirements for sensitivity and specificity of the prediction).The process can be automated so that if the threshold is exceeded at a given pixel in the image, it is indicated by color-coding in the image as indicated at process block 412

It should be apparent to those skilled in the art that other tissue mechanical properties can be measured using MRE techniques. As described in the above-cited U.S. Pat. No. 5,592,085, the disclosure of which is incorporated by reference, mechanical properties such as elasticity, viscosity and shear attenuation may be measured and imaged and used to detect disease conditions.

It should also be apparent that other imaging modalities which can detect mechanical properties of tissues may also be used to detect disease causing conditions. Ultrasound imaging methods such as that disclosed by M. Fatemi and J. F. Greenleaf “Vibro-Acoustography: An Imaging Modality Based On Ultrasound-Stimulated Acoustic Emission”, Proc. Natl. Acad. Sci. USA, Vol 96, pp 6603-08, June 1999 Engineering, may be used.

A variation of the above embodiment employs a mechanical characteristic criteria which looks to a change in liver stiffness as an indication of a disease causing condition. This procedure employs two stiffness measurements using the above-described MRE procedure. The first scan is performed after 6 hours of fasting and the second scan is performed 30 minutes after drinking a glucose or other solution that increases splanchnic blood flow. In normal individuals, liver stiffness does not significantly increase after eating. Deviations from this norm are detected by subtracting the stiffness values of corresponding pixels in the two images in process block 410 and the pixel locations demonstrating a significant increase in stiffness are indicated as possible disease causing conditions at process block 412. As before, color coding may be used to indicate the degree of increased stiffness and degree of disease causing conditions.

While such a subject's liver may not reveal any evidence of fibrosis, a condition which potentiates fibrosis may be present. Such subjects have faulty autoregulation of hepatic sinusoidal resistance, which causes intravascular pressure in the liver to rise when blood flow through the liver rises after eating. The elevated blood pressure causes stretching of cells throughout the liver for a transient period after meals. This stretching can trigger the development of hepatic fibrosis by causing transformation of stellate cells.

Another exemplary application of the present invention is the early detection of a condition in which breast cancer may occur. It is known that an increased radiographic breast density is associated with a higher lifetime risk of breast cancer. And yet, this association is not sufficiently specific to be routinely used to change the future management of the subject. The present invention may be used with such patients to determine if more aggressive screening for breast cancer is justified.

More specifically, the subject has an MRE or vibro-acoustic ultrasound examination of the breasts to measure the stiffness of fibro-glandular tissues. If the stiffness values are abnormally high, the presence of an extracellular matrix environment that, through mechanotransduction, increases the likelihood of malignant transformation of epithelial cells to cancer. These locations are indicated on the reconstructed image of the breast tissues. Such images not only alert the physician to increase the monitoring regimen, but also alert the physician to locations in the breast where tumors are likely to occur.

1. A method for detecting mechanical conditions in tissues that cause disease, the steps comprising:
a) performing an in vivo measurement of a mechanical property of the tissues;
b) detecting from the measurement a condition which may lead to a disease in the tissues; and
c) indicating the detected disease causing condition.

2. The method as recited in claim 1 in which the mechanical condition is elevated stiffness.

3. The method as recited in claim 1 in which the mechanical condition is an elevated change in stiffness

4. The method as recited in claim 1 in which step a) is performed by acquiring MRE data from the tissues using a magnetic resonance imaging system.

5. The method as recited in claim 4 in which step b) includes:
b)i) reconstructing an image of the tissues from the acquired MRE data;
b)ii) calculating a mechanical characteristic of the tissues at each image pixel; and
b)iii) comparing the calculated mechanical characteristic to a pre-established norm.

6. The method as recited in claim 5 in which the mechanical characteristic is tissue stiffness.

7. The method as recited in claim 5 in which the step c) includes displaying an image of the tissues in which pixel locations which have values of the mechanical characteristic that deviate from the norm are indicated.

8. The method as recited in claim 7 in which the pixel locations in the tissue image are color coded to indicate the degree of deviation from the norm.

9. The method as recited in claim 1 in which the tissues are located in an organ and the disease is fibrosis.

10. The method as recited in claim 9 in which the organ is the liver.

11. The method as recited in claim 1 in which the mechanical condition is stiffness and the disease is cancer.
